(19) United States
(12) Patent Application Publication
(10) Pub. No.: US 2004/0180384 A1
(43) Pub. Date: Sep. 16, 2004

(54) Test preparation for microscopes

(75) Inventors: Joerg Schaffer, Goettingen (DE); Hubert Bauch, Rosdorf (DE)

Correspondence Address: Gerald H. Kiel, Esq., REED SMITH LLP, 599 Lexington Avenue, New York, NY, 10022-7650, US

(73) Assignee: CARL ZEISS JENA GmbH

(21) Appl. No.: 10/734,903

(22) Filed: Dec. 12, 2003

(30) Foreign Application Priority Data
Dec. 13, 2002 (DE) 102 58 989.5

(51) Int. Cl. G01N 33/53; G01N 33/567; G01N 33/48; G01N 1/30

(52) U.S. Cl. 435/7.2; 435/40.5

(57) ABSTRACT

The invention is directed to a novel test preparation for microscopes, particularly fluorescence microscopes, which is characterized in that spectral excitation can be carried out in a freely selectable wavelength range of visible light.

### CROSS-REFERENCE TO RELATED APPLICATIONS

This application claims priority of German Application No. 102 58 989.5, filed Dec. 13, 2002, the complete disclosure of which is hereby incorporated by reference.

### BACKGROUND OF THE INVENTION

a) Field of the Invention

The subject matter of the invention is a test preparation for microscopes which has fluorescence characteristics and can be used to detect the function and/or performance of microscopes.

b) Description of the Related Art

Preparations which either show a certain autofluorescence or are subjected to special treatment with dyes have been used formerly in microscopy as fluorescence test preparations.

Synthetic preparations (e.g., spherical plastic bodies in sizes ranging from nanometers to micrometers) which essentially take on the fluorescence characteristics of the dye when mixed with dye molecules during manufacture are known. Such microspheres or microbeads, as they are called, are known, e.g., from U.S. Pat. No. 4,336,173, U.S. Pat. No. 4,247,434, U.S. Pat. No. 4,714,682, U.S. Pat. No. 4,868,126 and others.

Test preparations comprising biological tissue which is visible by staining either through illumination with natural light or by observing the fluorescence emission of the dyestuff with a microscope with adapted optical filters are also known. Further, preparations are used in which special functional groups of molecules or even tissues are fluorescence-labeled in a specific manner in that those dye molecules that are introduced are fixated by chemical bonding specifically to the functional groups and make it possible to identify them. Such test preparations are produced and sold, e.g., by the firm Molecular Probes, Eugene, Oreg., USA under the trade name FluoCells.

Finally, there are also naturally occurring tissue types with a certain autofluorescence. One such tissue is lily of the valley (convallaria majalis), whose stem cross sections are used to produce test preparations for laser scanning microscopes because of their pronounced three-dimensional honeycomb structure.

All known types of fluorescence preparations have a number of disadvantages: First, production using special dyes is complicated. Particularly labeling by means of special chemical bonding requires a high level of knowledge about the labeled specimen itself. Not all specimens can be labeled in this way with all dyes. Thus cell nuclei require different dyes than, e.g., actin. A plurality of chemical synthesis steps may have to be carried out to produce the final preparation. However, the chief disadvantage is that the fluorescence excitation and fluorescence emission of the preparations is a product of the corresponding characteristics of the dye molecules that are used, i.e., for every type of dye there exists only a narrow spectral range in which the specimen can be excited by light and, in addition, only a limited spectral region in which the fluorescence emission is carried out. These regions are usually in the order of magnitude of some 10 nanometers on the wavelength scale. These spectral regions are very limited even when there is autofluorescence and are dependent on the tissue or cell bond upon which they are based. Accordingly, only certain optical filters can be used to image the specimen. Therefore, different preparations are also needed to test different filter sets.

### OBJECT AND SUMMARY OF THE INVENTION

It is the primary object of the invention to overcome the disadvantages of the prior art and to provide a test preparation having many uses.

According to the invention, a test preparation for microscopes, particularly optical microscopes, comprises an object carrier and a biological cell bond arranged on the object carrier, wherein the cell bond is fixed under treatment by a compound which enables a freely selectable fluorescence excitation in a wavelength region with a breadth greater than 100 nm. Surprisingly, it has been shown that cell bonds which are fixated on the object carrier using glutardialdehyde (pentane dialdehyde) have a very broad fluorescence spectrum. The excitation and emission of fluorescence can be achieved over the entire spectral range of near ultraviolet (around 350 nm) to the visible region (about 700 nm). Accordingly, any desired combination of filter sets can be used to ensure imaging of this preparation.

The preparation according to the invention can advantageously be applied in fluorescence microscopy for calibration of optical processes such as confocal microscopy, widefield epifluorescence and methods of structured illumination. It has been shown, in addition, that the preparation according to the invention exhibits only a slight tendency to bleach out.

### DESCRIPTION OF THE PREFERRED EMBODIMENTS

The invention will be described in the following with reference to a preferred embodiment example. The preparation itself comprises animal or human tissue. The cell structure and tissue structure is fixated with glutardialdehyde. The concentration of glutardialdehyde and the fixating time depend on the type of tissue or cell. Typical values are 2% to 5% glutardialdehyde in PBS (phosphate-buffered saline solution) with an action period of 30 minutes. As a result of fixating, the proteins in the tissue are denatured through a chemical reaction with the glutardialdehyde and the structural cohesion of the specimen is accordingly ensured. The excess glutardialdehyde is then removed from the tissue by several washing steps. In order to produce sections from the tissue for microscopic observation, the tissue is worked up further. For example, it can be cryo-shocked or successively embedded in paraffin. A tissue block is formed which can be cut by a suitable cutting device (e.g., microtom). The thickness of the section is not fixed; typical section thicknesses are 2 to 20 μm. A rather thin section with a thickness of less than 10 μm is particularly suitable for some applications such as structured illumination.

The sections are arranged on a glass object carrier, preferably with standard dimensions (e.g., 26 mm*76 mm). The adhesion of the sections to the object carrier can be increased by coating the object carrier, e.g., with poly-D-lysine.

A specimen produced in this way has the broad spectral fluorescence characteristics found as a result of the invention. It is possible to add an antifading reagent to the embedding medium which prevents excessive fluorescence bleaching. As a rule, these reagents work by means of the bonding of free oxygen radicals which can consequently not destroy the chromophore groups of the present molecules. One example is produced and sold by the firm Molecular Probes under the tradename ProLong.

The preparation is protected against environmental influences by a cover glass, preferably of a standard thickness (0.17 mm), and can be preserved indefinitely. In this way, in addition, it is made accessible for observation with standard objectives with cover glass correction.

For some applications (e.g., the method of structured illumination), it is advantageous to use dense tissue structures in order to allow imaging over the entire visual field of the optical instrument and, at the same time, to ensure an image with as few gaps as possible. In other applications (e.g., confocal microscopy), it is possible to use rather fine structures in order to be able to document specific features of the method.

It may be advantageous to seal the preparation at the edge of the cover glass with a clear lacquer.

The invention is not limited to the described embodiment example; other chemical compounds which result in a sufficiently broad spectral fluorescence excitation/emission property of the fixated tissue can also be used.

While the foregoing description and drawings represent the present invention, it will be obvious to those skilled in the art that various changes may be made therein without departing from the true spirit and scope of the present invention.

What is claimed is:

1. A test preparation for microscopes, particularly optical microscopes, comprising:
an object carrier; and
a biological cell bond arranged on the object carrier, wherein the cell bond is fixed under treatment by a compound which enables a freely selectable fluorescence excitation in a wavelength region with a breadth of the order of 100 nm or greater.

2. The test preparation for microscopes according to claim 1, wherein the cell bond is fixed under treatment by a compound which enables a freely selectable fluorescence excitation in a wavelength range from 450 to 650 nm.

3. The test preparation for microscopes according to claim 1, wherein the cell bond is fixed under treatment by a compound which enables a freely selectable fluorescence excitation in a wavelength range from 350 to 700 nm.

4. The test preparation for microscopes according to claim 1, wherein the cell bond is fixated using glutardialdehyde.

5. The test preparation for microscopes according to claim 1, wherein an antifading reagent is added to the compound.

6. The test preparation for microscopes according to claim 1, wherein the cell bond has a dense structure over the entire visual field of the microscope.
